(19) United States
(12) Patent Application Publication
(10) Pub. No.: US 2023/0157300 A1
(43) Pub. Date: May 25, 2023

(54) ANTI-VIRAL AGENT

(71) Applicant: YOO CORPORATION, Yao-shi, Osaka (JP)

(72) Inventors: Mami WAKAMA, Yao-shi, Osaka (JP); Masami YOKOTA, Yao-shi, Osaka (JP); Yosuke WAKAMA, Yao-shi, Osaka (JP); Mika SUEYOSHI, Yao-shi, Osaka (JP); Satoshi OKAMOTO, Osaka-shi, Osaka (JP); Hiroyuki SAKITA, Osaka-shi, Osaka (JP)

(21) Appl. No.: 17/921,457

(22) PCT Filed: Feb. 16, 2022
(86) PCT No.: PCT/JP2022/006097

§ 371 Date: Oct. 26, 2022

(30) Foreign Application Priority Data
Mar. 19, 2021 (JP) 2021-046234

(51) Int. Cl. A01N 59/20; A01N 59/26; A61K 8/29; A61Q 17/00; A01P 1/00; C11D 3/48; A61K 8/25

(52) U.S. Cl. CPC A01N 59/20; A01N 59/26; A61K 8/29; A61Q 17/005; A01P 1/00; C11D 3/48; A61K 8/25; A61K 2800/524

(57) ABSTRACT

The present invention aims to provide an antiviral agent that is safe to a human body, and provides an antiviral agent comprising a titanium phosphate compound, a silicic acid compound, a silver compound, and a copper compound.

### TECHNICAL FIELD

The present invention relates to an antiviral agent.

### BACKGROUND ART

The spread of the novel coronavirus (COVID-19) has prompted new lifestyle practices. It is considered important to use disinfectants or the like that exhibit antiviral effects, and such disinfectants are often provided at the entrances of retail stores and public facilities. Anti-viral measures are being actively applied in a variety of places, not only in retail stores and public facilities, but also inside buses, trains, and other vehicles.

Patent Literature 1 discloses a composition comprising titania phosphate. Specifically, it discloses that the composition has antibacterial and antifungal effects. Patent Literature 2 discloses a composition comprising silver nitrate, boric acid, zinc nitrate, and copper nitrate together with hydrolyzed titanium tetrachloride. Specifically, it discloses that the composition has deodorant effects, antibacterial effects, antifungal effects, antiviral effects, and effects of preventing marine organism adhesion.

### CITATION LIST

### Patent Literature

PTL 1: Japanese Patent No. 3829640

PTL 2: Japanese Patent No. 6012345

### SUMMARY OF INVENTION

### Technical Problem

PTL 1 nowhere teaches that the composition of PTL 1 exhibits antiviral effects. PTL 2 teaches that the composition containing hydrolyzed titanium tetrachloride exhibits antiviral effects; however, the composition contains a large amount of zinc nitrate, which may have an adverse effect on the human body, thus raising concerns about its use as an antiviral agent.

### Solution to Problem

To solve the above problem, the present inventor conducted extensive research, and consequently, found that the composition containing a titanium phosphate compound, a silicic acid compound, a silver compound, and a copper compound exhibits antiviral effects. The present inventor also found that by mixing a silicic acid compound, a silver compound, and a copper compound, in this order, in a solution containing a titanium phosphate compound, a transparent composition can be produced. The present invention was completed based on these findings, and widely includes the following embodiments of the invention.

Item 1. An antiviral agent comprising a titanium phosphate compound, a silicic acid compound, a silver compound, and a copper compound.
Item 2. The antiviral agent according to Item 1, wherein the titanium phosphate compound is a compound represented by

Ti(OH)ₓ(PO₄)_{y}(HPO₄)_{z}(H₂PO₄)ₗ(OR)ₙ,

wherein
R represents a C₁₋₄ alkyl group,
x is 0, 1, 2, or 3,
y is 0, 1, 2, 3, or 4,
z is 0, 1, 2, 3, or 4,
l is 0, 1, 2, 3, or 4, and
m is 0, 1, 2, or 3,
with the proviso that x+3y+2z+l+m=4 and y+z+l≥1 are satisfied; and/or
a condensate of the compound.
Item 3. The antiviral agent according to Item 1 or 2, wherein the silicic acid compound is at least one compound selected from the group consisting of orthosilicic acid, metasilicic acid, metadisilicic acid, polymers thereof, and salts thereof.
Item 4. The antiviral agent according to any of Items 1 to 3 above, wherein the silver compound is at least one compound selected from the group consisting of silver nitrate, silver oxide, silver sulfide, silver bromide, and silver iodide.
Item 5. The antiviral agent according to any of Items 1 to 4 above, wherein the copper compound is at least one compound selected from the group consisting of copper nitrate, copper oxide, copper sulfide, copper sulfate, and silver chloride.
Item 6. The antiviral agent according to any one of Items 1 to 5, wherein the antiviral agent is against at least one virus selected from the group consisting of viruses belonging to the family Caliciviridae, viruses belonging to the family Orthomyxoviridae, viruses belonging to the family Coronaviridae, and viruses belonging to the family Adenoviridae.
Item 7. The antiviral agent according to Item 6, wherein the virus belonging to the family Caliciviridae is at least one virus selected from the group consisting of viruses belonging to the genus Vesivirus, viruses belonging to the genus Lagovirus, viruses belonging to the genus Norovirus, viruses belonging to the genus Sapovirus, and viruses belonging to the genus Nebovirus.
Item 8. The antiviral agent according to Item 6, wherein the virus belonging to the family Orthomyxoviridae is at least one virus selected from the group consisting of viruses belonging to the genus Alphainfluenzavirus, viruses belonging to the genus Betainfluenzavirus, viruses belonging to the genus Gammainfluenzavirus, viruses belonging to the genus Deltainfluenzavirus, viruses belonging to the genus Isavirus, viruses belonging to the genus Quaranjavirus, and viruses belonging to the genus Thogotovirus.
Item 9. The antiviral agent according to Item 6, wherein the virus belonging to the family Coronaviridae is at least one virus selected from the group consisting of viruses belonging to the genus Alphacoronavirus, viruses belonging to the genus Betacoronavirus, viruses belonging to the genus Gammacoronavirus, viruses belonging to the genus Deltacoronavirus, viruses belonging to the subgenus Merbecovirus, and viruses belonging to the subgenus Sarvecovirus.
Item 10. The antiviral agent according to Item 6, wherein the virus belonging to the family Adenoviridae is at least one virus selected from the group consisting of viruses belonging to the genus Atadenovirus, viruses belonging to the genus fowl Adenovirus, viruses belonging to the genus Ichtadenovirus, viruses belonging to the genus Mastadenovirus, and viruses belonging to the genus Siadenovirus.
Item 11. The antiviral agent according to any one of Items 1 to 10, wherein the antiviral agent is against any one of feline calicivirus, murine norovirus, influenza A virus (H3N2), human coronavirus strain 229E, SARS coronavirus (SARS-CoV-2), and adenovirus type 5.
Item 12. The antiviral agent according to any one of Items 1 to 11, which is used for cosmetics, disinfectants, or detergents.
Item 13. A method for producing the antiviral agent according to any one of Items 1 to 12, the method comprising mixing a silicic acid compound, a silver compound, and a copper compound in this order, in a liquid containing a titanium phosphate compound.

### Advantageous Effects of Invention

The antiviral agent of the present invention can be used safely in the human body. The antiviral agent produced by the method for producing the antiviral agent of the present invention is transparent. Accordingly, the antiviral agent has an effect of not coloring a target to be used when it is used in the field of cosmetics, disinfectants, or detergents. In addition, since the antiviral agent produced by the production method of the present invention does not contain a precipitate etc., it has an effect of not clogging an instrument such as a sprayer when used.

### BRIEF DESCRIPTION OF DRAWINGS

FIG. 1 shows photographs of antiviral agents of Production Example 1 (M052120K) and Comparative Example 1 (M072114K) of the present invention.

### DESCRIPTION OF EMBODIMENTS

The term “include” or “contain” as used in the following specification encompasses the meanings of consisting essentially of and consisting of.

The contents of the references and webpages listed in this specification may be incorporated herein by reference.

The term “antiviral effects” in the present specification is not interpreted as having a meaning significantly departing from “antiviral effects” as used by those skilled in the art. For example, such effects include the effects of preventing viral infection of the human body, preventing viral entry into cells, inhibiting self-replication of viruses that have entered cells, preventing adverse effects caused after viral entry into cells, and destroying the structure of a virus itself.

### Antiviral Agent

The antiviral agent of the present invention contains a titanium phosphate compound, silicic acid compound, silver compound, and copper compound.

The titanium phosphate compound contained in the antiviral agent of the present invention is not particularly limited as long as it exhibits the effects of the present invention, and a variety of known titanium phosphate compounds can be used. For example, one, two, or more compounds represented by the following formula: Ti(OH)ₓ(PO₄)_{y}(HPO₄)_{z}(H₂PO₄)ₗ(OR)ₙ can be used as the titanium phosphate compound.

R indicates a C₁₋₄ alkyl group. x is 0, 1, 2, or 3. y is 0, 1, 2, 3, or 4. z is 0, 1, 2, 3, or 4. l is 0, 1, 2, 3, or 4. m is 0, 1, 2, or 3. Note that x+3y+2z+l+m=4 and y+z+l≥1 are satisfied.

The titanium phosphate compound contained in the antiviral agent of the present invention can include a condensate thereof. The condensate of the titanium phosphate compound is not particularly limited, and can be, for example, a compound in which about 2 to 10 molecules of the above titanium phosphate compound are condensed.

The condensation form of the condensate is not particularly limited, and can be, for example, the condensation form obtained by the removal of a water molecule from the titanium phosphate compound. The condensate can be condensed by a single type of a titanium phosphate compound or by two or more different types of titanium phosphate compounds.

The titanium phosphate compound can be produced, for example, by the production method of Patent Literature 1. Specifically, the titanium phosphate compound can be obtained by reacting the hydrolysate of titanium tetrachloride with phosphoric acid. Specifically, the titanium phosphate compound can also be referred to as “phosphorylation of titanium tetrachloride hydrolysate.”

The amount of the titanium phosphate compound contained in the antiviral agent of the present invention can be suitably selected from a wide range of amounts in which the effects of the present invention are attained. The amount of the titanium phosphate compound is usually about 1 to 70 wt %, and preferably about 5 to 60 wt %, based on the total amount of 100 wt % of the antiviral agent of the present invention.

As the silicic acid compound contained in the antiviral agent of the present invention, a known silicic acid compound can be widely used. Examples include the compound represented by the following formula: [SiOₓ(OH)₄₋₂ₓ]ₙ or salt thereof. The compounds represented by the above formula or salts thereof may be used singly or in a combination of two or more as the silicic acid compound.

x and n in the formula are arbitrary numbers.

Specific examples of the silicic acid compound contained in the antiviral agent of the present invention include orthosilicic acid, metasilicic acid, metadisilicic acid, polymers thereof, and salts thereof. Among these silicic acid compounds, sodium metasilicate or salts thereof are preferred in view of their high solubility in water.

The amount of the silicic acid compound contained in the antiviral agent of the present invention can be suitably selected from a wide range of amounts in which the effects of the present invention are attained. For example, the amount of the silicic acid compound is usually about 0.001 to 40 wt %, and preferably about 0.01 to 20 wt %, based on the total amount of 100 wt % of the antiviral agent of the present invention.

A wide variety of known silver compounds can be used as a silver compound contained in the antiviral agent of the present invention. Examples include silver nitrate, silver oxide, silver sulfide, silver bromide, and silver iodide. Among these silver compounds, silver nitrate is preferred in view of its high solubility in water. These silver compounds can be used singly or in a combination of two or more as the silver compound.

The amount of the silver compound contained in the antiviral agent of the present invention can be suitably selected from a wide range of amounts in which the effects of the present invention are attained. For example, the amount of the silver compound is usually about 0.001 to 40 wt %, and preferably about 0.01 to 20 wt %, based on the total amount of 100 wt % of the antiviral agent of the present invention.

A wide variety of known copper compounds can be used as the copper compound. Specific examples include copper nitrate, copper oxide, copper sulfide, copper sulfate, and copper chloride. Copper nitrate is preferred in view of its high solubility in water. These copper compounds can be used singly or in a combination of two or more as the copper compound.

The amount of the copper compound contained in the antiviral agent of the present invention can be suitably selected from a wide range of amounts in which the effects of the present invention are attained. For example, the amount of the copper compound is usually about 0.001 to 80 wt %, and preferably about 0.01 to 50 wt %, based on the total amount of 100 wt % of the antiviral agent.

The antiviral agent of the present invention can exhibit antiviral effects even when a zinc compound such as zinc nitrate, which has adverse effects on the human body, is not contained, or is contained in a small amount. The amount of such a zinc compound is not particularly limited as long as it does not impair the effects of the present invention. The amount of the zinc compound is, for example, usually about 0 to 4 wt %, and preferably about 0 to 3 wt %, based on the total amount of 100 mass % of the antiviral agent of the present invention.

Viruses against which the antiviral agent of the present invention exhibits antiviral effects are not particularly limited. Examples include viruses belonging to the family Caliciviridae, viruses belonging to the family Orthomyxoviridae, viruses belonging to the family Coronaviridae, and viruses belonging to the family Adenoviridae.

The family Caliciviridae is the generic name for viruses with a genome of plus single-stranded RNA, and is one of the Nidovirales.

Such viruses belonging to the family Caliciviridae are not particularly limited. Examples include viruses belonging to the genus Vesivirus, viruses belonging to the genus Lagovirus, viruses belonging to the genus Norovirus, viruses belonging to the genus Sapovirus, and viruses belonging to the genus Nebovirus.

Typical examples of viruses belonging to the genus Vesivirus include feline calicivirus or vesicular exanthema virus. Typical examples of viruses belonging to the genus Lagovirus include rabbit haemorrhagic disease virus or European brown hare syndrome virus. Typical examples of a virus belonging to the genus Norovirus include Norwalk virus. Typical examples of the virus belonging to the genus Sapovirus include Sapporo virus.

Of the viruses belonging to the family Caliciviridae listed above, mouse norovirus belonging to the genus Norovirus or feline calicivirus belonging to the genus Vesivirus is preferred.

The family Orthomyxoviridae is a generic term for viruses having an envelope in which a minus single-stranded RNA is a genome.

Viruses belonging to the family Orthomyxoviridae are not particularly limited. Examples include viruses belonging to the genus Alphainfluenzavirus, viruses belonging to the genus Betainfluenzavirus, viruses belonging to the genus Gammainfluenzavirus, viruses belonging to the genus Deltainfluenzavirus, viruses belonging to the genus Isavirus, viruses belonging to the genus Quaranjavirus, and viruses belonging to the genus Thogotovirus.

Among the viruses belonging to the family Orthomyxoviridae listed above, viruses belonging to the genus Alphainfluenzavirus are preferred. Influenza A virus (H3N2) belonging to the genus Alphainfluenzavirus is more preferred as a virus belonging to the family Orthomyxoviridae.

The family Coronaviridae, also called the subfamily Orthocoronavirus, is one of the families belonging to the Nidovirinae, and is the generic name for viruses having an envelope in which a plus single-stranded RNA is a genome.

Such viruses belonging to the family Coronaviridae are not particularly limited, and examples include viruses belonging to the genus Alphacoronavirus, viruses belonging to the genus Betacoronavirus, viruses belonging to the genus Gammacoronavirus, viruses belonging to the genus Deltacoronavirus, viruses belonging to the subgenus Merbecovirus, and viruses belonging to the subgenus Sarvecovirus.

Among viruses belonging to the family Coronaviridae listed above, preferable examples include viruses belonging to the genus Alphacoronavirus, viruses belonging to the subgenus Merbecovirus, and viruses belonging to the subgenus Sarvecovirus, and more preferable examples include coronavirus 229E belonging to the genus Alphacoronavirus and Covid-19 (SARS-CoV-2) belonging to the subgenus Sarvecovirus.

The family Adenoviridae is a generic term for viruses having an envelope in which a double-stranded DNA is a genome.

Viruses belonging to the family Adenoviridae are not particularly limited. Examples include viruses belonging to the genus Atadenovirus, viruses belonging to the genus fowl adenovirus, viruses belonging to the genus Ichtadenovirus, viruses belonging to the genus Mastadenovirus, and viruses belonging to the genus Siadenovirus

Among the viruses belonging to the family Adenoviridae listed above, Mastadenovirus is preferred. Adenovirus type 5 can be listed as a more preferable example of the virus belonging to the family Adenovirus.

The pH of the antiviral agent of the present invention is not particularly limited as long as the antiviral effects are exhibited, and it can be suitably set to a pH applicable to the applied target. For example, the pH of the antiviral agent can be set to 1.5 to 10. In view of avoiding damaging the applied target and avoiding the precipitation of the antiviral agent of the present invention, the pH of the antiviral agent is preferably 2 to 6.

The antiviral agent of the present invention can be diluted for use; however, diluting the antiviral agent of the present invention with water or the like will tend to increase its pH. The dilution rate is not particularly limited as long as it does not inhibit the effect exerted by the antiviral agent of the present invention, and the antiviral agent of the present invention can be diluted 10 to 50 times.

The antiviral agent of the present invention can be widely used in a field in which antiviral effects are required. Examples of such fields include cosmetics, disinfectants, and detergents.

The antiviral agent of the present invention may contain other components together with a titanium phosphate compound, a silicic acid compound, a silver compound, and a copper compound to such an extent that the effect of the present invention is not impaired. Such other components are not particularly limited, and examples include a carrier, base, solvent, dispersant, emulsifier, buffer, chelating agent, stabilizer, excipient, binder, disintegrant, lubricant, thickener, moisturizer, colorant, flavoring agent, and medium.

The form of the antiviral agent of the present invention can be suitably determined according to the field in which it is used.

When the antiviral agent of the present invention is used as a cosmetic, examples include make-up cosmetics, such as foundation, cheek, and powder; basic cosmetics, such as toner, emulsion, skin cream, lotion, oil, and pack; skin cleansing products, such as facial cleansers, cleansers, and body soaps; hair cosmetics, such as shampoo, rinse, conditioner, hair dressing products, and hair growth products; bath additives, such as bath salts, bath tablets, and bath liquids; massaging agents; and cleaning agents.

When the antiviral agent of the present invention is used as a disinfectant or detergent, it can be, for example, in the form of liquid, suspension, spray, gel, lotion, emulsion, ointment, or stick.

When the antiviral agent of the present invention is used as a cosmetic, the applied target is a human. Specific examples include human skin or scalp.

When the antiviral agent of the present invention is used as a disinfectant or detergent, it can be applied to a human and an animal, as well as any other targets for which the antiviral effects are desired.

The target to which the antiviral agent is applied, other than humans and animals, is not particularly limited. Examples include office machines, home appliances, air-conditioning equipment, vacuum cleaners, desks, chairs, sofas, benches, windows, walls, floors, ceilings, suspension leather, handles, seats, automatic ticket gates, ticket vending machines, vending machines, doors, fences, handrails, tableware, cooking utensils, packaging film, packaging bags, jars, bottles, packaging packs, sinks, toilet bowls, stationery, books, shelves, toothbrushes, mirrors, air conditioning filters, masks, coats, jackets, trousers, skirts, hospital gowns, lab coats, surgical gowns, shirts, knitted shirts, blouses, sweaters, cardigans, nightwear, underwear, underclothes, diapers, supporters, socks, tights, stockings, hats, scarves, mufflers, neck scarves, stoles, gloves, clothing lining, clothing interlining, clothing padding, working clothes, uniforms, school uniforms, and other clothing; curtains, screen doors, duvet fabric, duvet cotton, duvet covers, pillowcases, sheets, mats, carpets, towels, handkerchiefs, wall cloth, adhesive bandages, and bandages.

The amount of the antiviral agent of the present invention cannot be unconditionally determined because it depends on the object of use, purpose of use, and environment of use. For example, if the antiviral agent is used for walls, the amount of the antiviral agent of the present invention can be about 15 to 20 cc/m².

### Method for Producing Antiviral Agent

The antiviral agent of the present invention can be produced by mixing a silicic acid compound, a copper compound, and a silver compound, in this order, in a solution containing a titanium phosphate compound.

According to the method for producing the antiviral agent of the present invention, a transparent antiviral agent having almost no precipitation can be produced. Accordingly, the antiviral agent produced by the production method of the present invention has an effect of not coloring a target on which it is used. The production method of the present invention also has advantages such as preventing clogging of a sprayer or the like with which the antiviral agent of the present invention is used.

The titanium phosphate compound in the production method is as explained in the antiviral agent section.

The liquid (solvent) containing a titanium phosphate compound is not particularly limited as long as the effect of the present invention is not impaired. Specific examples include water used in the dilution of a titanium phosphate compound, which may further contain alcohol.

The alcohol is not particularly limited as long as the effect of the present invention is achieved. Specific examples include C₁₋₄ alcohols, such as ethanol, methanol, n-propyl alcohol, isopropyl alcohol, n-butyl alcohol, isobutyl alcohol, sec-butyl alcohol, and tert-butyl alcohol. Of these alcohols, ethanol is preferred from the viewpoint of odor.

The silicic acid compound, zinc compound, copper compound, and silver compound used in the production method of the present invention are as described in the antiviral agent section above. These compounds can be dispersed in the same solvent as the titanium phosphate compound.

The amounts of the titanium phosphate compound, silicic acid compound, zinc compound, copper compound, and silver compound used in the production method of the present invention are as described in the antiviral agent section.

The temperature during mixing in the production method of the present invention is not particularly limited, and can be a room temperature of about 0 to 40° C. After the above mixing step, a stirring step may be included. If the precipitate is present in the obtained antiviral agent, a known solid-liquid separation step to remove such a precipitate can be used.

### EXAMPLES

The following describes examples to illustrate the details of the antiviral agent of the present invention. Of course, the present invention is not limited to the following examples.

### Production Example

A solution containing a titanium tetrachloride hydrolysate obtained by diluting 100 wt % of a titanium tetrachloride solution in 50 wt % of a mixed solution of water and ethanol to have a concentration of 33 wt % was further diluted 11 times in water, and 10 w % of a phosphoric acid was added thereto, thus obtaining a solution containing a titanium phosphate compound. 2 g of sodium silicate, 5 g of silver nitrate, and 5 g of copper nitrate were added in this order, and mixed at room temperature to produce an antiviral agent used in the following Example. The antiviral agent was a clear solution with a slightly bluish color and its pH was about 1.5. The antiviral agent thus produced is referred to below as the “antiviral agent of Production Example 1,” or “Production Example 1.”

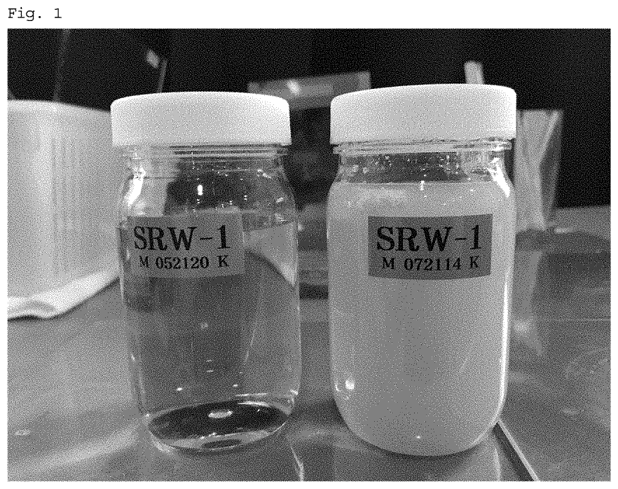

As Comparative Example, 2 g of sodium silicate, 5 g of copper nitrate, and 5 g of silver nitrate were mixed in this order in an aqueous solution containing a titanium phosphate compound. FIG. 1 shows photographic images of the antiviral agents of Production Example 1 and Comparative Example.

M052120K on the left in the photographic image shown in FIG. 1 is the antiviral agent of Production Example 1, and M072114K on the right is the antiviral agent of the Comparative Example. As is clear from the results, the Comparative Example on the right becomes cloudy due to precipitation, whereas the antiviral agent of Production Example 1 on the left can be transparent without precipitation.

Subsequently, the antiviral agent was diluted 30 times. The antiviral agent after dilution was transparent and had a pH of about 2.8. The antiviral agent after dilution may be sometimes referred to as the “antiviral agent of Production Example 2” or “Production Example 2.”

### Example 1

An experiment to confirm antiviral effects of an antiviral agent on human coronavirus 229E (family Coronaviridae), adenovirus type 5 (family Adenoviridae), and mouse norovirus (family Caliciviridae) was conducted according to UNI EN 14476+A2:2019 (http://store.uni.com/catalogo/en-14476-2013-a2-2019/). The antiviral agents used in this example are antiviral agents obtained by diluting the antiviral agent of Production Example 1 to 50, 60, and 97%. (The dilution to 50% and 60% is performed by hard water, and dilution to 97% is performed by diluted water.) The test was conducted using the following sample combination. An untreated experiment was also performed as a control.

1: Human coronavirus 229E (ATCC VR-740) against MRC-5 cells (ATCC CCL-171)
2: Adenovirus type 5 (ATCC VR-5) against Hela cells (ATCC CCL-2)
3: Mouse norovirus against Raw 264.7 cells (ATCC TIB-71) (Strain 599 Berlin Friedrich Loeffler Institute RVB-0651)

Other conditions are as follows. Cell growth medium: 10% FCS-containing MEM for all cells Cell maintenance medium: 2% FCS-containing MEM for all cells Interference material: bovine albumin (0.3 g/l) Infection conditions: 20° C. for 24 hours

The results of the antiviral activity value calculated from the virus titer measured using the Spearman-Kerber method are shown in Table 4 below. A numerical value of 2 or more is evaluated as having antiviral activity, and the higher the value is, the stronger the antiviral activity is. Note that “-” indicates no antiviral activity.

TABLE 1
Table 1

| | | Antiviral activity |
|---|---|---|
| Target virus | Concentrat ion | value |
| Human coronavirus 229E | Control | — |
| | 50% | >4 |
| | 60% | >4 |
| | 97% | >4 |
| Adenovirus type 5 | Control | — |
| | 50% | 2.625 |
| | 60% | 3.25 |
| | 97% | 2.875 |
| Mouse norovirus | Control | — |
| | 50% | 2.875 |
| | 60% | 3.125 |
| | 97% | 2.75 |

As shown in Table 1, it was revealed that the antiviral agent of Production Example 2 exhibited antiviral effects on human coronavirus 229E belonging to the family Coronaviridae, adenovirus type 5 belonging to the family Adenoviridae, and mouse norovirus belonging to the family Caliciviridae.

### Example 2

An experiment to confirm the effect of the antiviral agent of Production Example 2 on feline calicivirus belonging to the family Caliciviridae was conducted according to ISO 21702 (https://www.iso.org/standard/71365.html), which was a modified version of ISO 22196 (JIS Z 2801) (an antimicrobial test method for plastic products) for viruses. The test was conducted using the following samples.

Test virus: Feline calicivirus (F-9; ATCC VR-782)
Host cells: CRFK cells (ATCC CCL-94)
Infection conditions: 25° C., 24 hours
Washing liquid: 10% FBS-containing SCDLP medium

In this example, light resistance treatment according to the durability standard of the Society of International sustaining growth for Antimicrobial Articles was conducted as a pre-treatment. As a control, an untreated experiment was conducted. The results are shown in Table 2.

TABLE 2
Table 2

| Pre- | | | Virus infection |
|---|---|---|---|
| treatment | Sample | | value |
| Pre- | Untreated | Immediately | 6.25 pfu/cm² |
| treatment | | after | |
| | | administration | |
| | | After 24 hours | 5.10 pfu/cm² |
| | Production | After 24 hours | 1.34 pfu/cm² |
| | Example 2 | | |
| Without | Untreated | Immediately | 5.13 pfu/cm² |
| pre- | | after | |
| treatment | | administration | |
| | | After 24 hours | 4.81 pfu/cm² |
| | Production | After 24 hours | 2.41 pfu/cm² |
| | Example 2 | | |

The results shown in Table 2 revealed that the antiviral agent of Production Example 2 exhibited antiviral effects on feline calicivirus belonging to the family Caliciviridae, regardless of the pre-treatment.

### Example 3

An experiment to confirm the effect of the antiviral agent of Production Example 2 on the influenza A virus belonging to the family Orthomyxoviridae was conducted according to ISO21702. The test was conducted using the following samples.

Test virus: Influenza A virus (H3N2; ATCC VR-1679)
Host cells: MDCK cells (ATCC CCL-34)
Infection conditions: 25° C., 24 hours
Washing liquid: SCDLP medium

In this example, light resistance treatment according to the durability standard of the Society of International sustaining growth for Antimicrobial Articles was conducted as a pre-treatment. As a control, an untreated experiment was conducted. The results are shown in Table 2.

TABLE 3
Table 3

| Pre- | | | Virus infection |
|---|---|---|---|
| treatment | Sample | | value |
| Pre- | Control | Immediately | 4.96 pfu/cm² |
| treatment | | after | |
| | | administration | |
| | | After 24 hours | 4.16 pfu/cm² |
| | Production | After 24 hours | 2.54 pfu/cm² |
| | Example 2 | | |
| Without | Control | Immediately | 5.08 pfu/cm² |
| pre- | | after | |
| treatment | | administration | |
| | | After 24 hours | 4.14 pfu/cm² |
| | Production | After 24 hours | 1.66 pfu/cm² |
| | Example 2 | | |

The results shown in Table 3 revealed that the antiviral agent of Production Example 2 exhibited antiviral effects on the influenza A virus (H3N2) belonging to the family Orthomyxoviridae, regardless of the pre-treatment.

### Example 4

An experiment to confirm the effect of the antiviral agent of Production Example 2 on SARS corona virus belonging to the family corona virus was conducted according to ISO21702. The test was conducted using the following samples. The results are shown in Table 4 below.

Test virus: SARS-CoV-2 (Covid-19, JPN/TY/WK-521; National Institute of Infectious Diseases)
Host cells: VeroE6/TMPRSS2 (JVRB1819)
Cell culture liquid: DMEM or MEM
Infection conditions: 25° C., 24 hours
Washing liquid: Liquid obtained by diluting SCDLP in a 2% FBS-containing MEM to 10 times

TABLE 4
Table 4

| | | Virus infection |
|---|---|---|
| Sample | | value |
| Control | Immediately | 5.70 pfu/cm² |
| | after | |
| | administration | |
| | After 24 hours | 4.70 pfu/cm² |
| Production | After 24 hours | 0.80 pfu/cm² |
| Example 2 | | |

The results shown in Table 4 revealed that the antiviral agent of Production Example 2 had antiviral effects on Covid-19 belonging to the family Coronaviridae.

### Example 5

A safety test (acute inhalation toxicity test) was conducted on the above antiviral agents. 6.5 g of the antiviral agent of Production Example 2 was inhaled by spraying to 4-week-old ICR mice (5 males and 5 females), and then body weight and other parameters were observed. 14 days later, the weight of the males increased by an average of 6.48 g, while the weight of the females increased by an average of 4.70 g.

The appearance of the body surface, guanine, intracranial cavity, thoracic cavity, intra-abdominal organs, and lymph nodes of the mice 14 days after inhalation was visually confirmed. As a result, no abnormalities were observed in either sex.

These results clearly showed that the antiviral agent of Production Example 1 was safe for living organisms.

1. An antiviral agent comprising a titanium phosphate compound, a silicic acid compound, a silver compound, and a copper compound.

2. The antiviral agent according to claim 1, which satisfies a constituent element according to any one of [1] to [4] below:
[1] the titanium phosphate compound is a compound represented by
Ti(OH)ₓ(PO₄)_{y}(HPO₄)_{z}(H₂PO₄)ₗ(OR)ₘ,
wherein
R represents a C₁₋₄ alkyl group,
x is 0, 1, 2, or 3,
y is 0, 1, 2, 3, or 4,
z is 0, 1, 2, 3, or 4,
l is 0, 1, 2, 3, or 4, and
m is 0, 1, 2, or 3,
with the proviso that x+3y+2z+l+m=4 and y+z+l≥1 are satisfied; and/or
a condensate of the compound,
[2] the silicic acid compound is at least one compound selected from the group consisting of orthosilicic acid, metasilicic acid, metadisilicic acid, polymers thereof, and salts thereof,
[3] the silver compound is at least one compound selected from the group consisting of silver nitrate, silver oxide, silver sulfide, silver bromide, and silver iodide, and
[4] the copper compound is at least one compound selected from the group consisting of copper nitrate, copper oxide, copper sulfide, copper sulfate, and silver chloride.

3. The antiviral agent according to claim 1, wherein the antiviral agent is against any one of viruses belonging to the family Caliciviridae, viruses belonging to the family Orthomyxoviridae, viruses belonging to the family Coronaviridae, and viruses belonging to the family Adenoviridae.

4. The antiviral agent according to claim 3, which satisfies a constituent element according to any one of (1) to (4) below:
(1) the virus belonging to the family Caliciviridae is at least one virus selected from the group consisting of viruses belonging to the genus Vesivirus, viruses belonging to the genus Lagovirus, viruses belonging to the genus Norovirus, viruses belonging to the genus Sapovirus, and viruses belonging to the genus Nebovirus,
(2) the virus belonging to the family Orthomyxoviridae is at least one virus selected from the group consisting of viruses belonging to the genus Alphainfluenzavirus, viruses belonging to the genus Betainfluenzavirus, viruses belonging to the genus Gammainfluenzavirus, viruses belonging to the genus Deltainfluenzavirus, viruses belonging to the genus Isavirus, viruses belonging to the genus Quaranjavirus, and viruses belonging to the genus Thogotovirus,
(3) the virus belonging to the family Coronaviridae is at least one virus selected from the group consisting of viruses belonging to the genus Alphacoronavirus, viruses belonging to the genus Betacoronavirus, viruses belonging to the genus Gammacoronavirus, viruses belonging to the genus Deltacoronavirus, viruses belonging to the subgenus Merbecovirus, and viruses belonging to the subgenus Sarvecovirus, and
(4) the virus belonging to the family Adenoviridae is at least one virus selected from the group consisting of viruses belonging to the genus Atadenovirus, viruses belonging to the genus fowl Adenovirus, viruses belonging to the genus Ichtadenovirus, viruses belonging to the genus Mastadenovirus, and viruses belonging to the genus Siadenovirus.

5. The antiviral agent according to claim 1, wherein the antiviral agent is against any one of feline calicivirus, murine norovirus, influenza A virus (H3N2), human coronavirus strain 229E, SARS coronavirus (SARS-CoV-2), and adenovirus type 5.

6. The antiviral agent according to claim 1, which is used for cosmetics, disinfectants, or detergents.

7. A method for producing the antiviral agent according to claim 1, the method comprising mixing the silicic acid compound, the silver compound, and the copper compound in this order, in a liquid containing the titanium phosphate compound.
